(19) United States
(12) Patent Application Publication
(10) Pub. No.: US 2023/0310133 A1
(43) Pub. Date: Oct. 5, 2023

(54) DEVICE FOR NEEDLE-FREE INJECTION OF A FLUID INTO AN ANIMAL

(71) Applicant: Henke-Sass, Wolf GmbH, Tuttlingen (DE)

(72) Inventors: Frank ALTERMANN, Tuttlingen (DE); Maikel WIEDMANN, Immendingen (DE); Robin SAUTER, Tuttlingen (DE); Anika SCHMIDT, Fridingen (DE)

(21) Appl. No.: 18/127,646

(22) Filed: Mar. 28, 2023

(30) Foreign Application Priority Data
Mar. 29, 2022 (DE) 102022107416.1

(51) Int. Cl. A61D 7/00; A61M 5/30

(52) U.S. Cl. CPC A61D 7/00; A61M 5/3007; A61M 2005/3022; A61M 2205/332; A61M 2205/3327; A61M 2250/00

(57) ABSTRACT

A device for the needle-free injection of a fluid into an animal can include a contact device including a shaped contact surface that is shaped in conformity with a body part of the animal to which the injection is to be administered. Also included is a first injector including a front dispensing end which, during the needle-free injection, extends through a first passage opening in the shaped contact surface and protrudes in relation to the shaped contact surface in a region adjacent to the first passage opening, a first measuring device which outputs at least one first measuring signal in order to detect contact of the animal with the front dispensing end of the first injector, and a control unit which activates a needle-free injection via the first injector on the basis of the at least one first measuring signal.

### PRIORITY

This application claims the benefit of German Patent Application No. 10 2022 107 416.1, filed on 29 Mar. 2022, which is hereby incorporated herein by reference in its entirety.

### FIELD

The present invention relates to a device for the needle-free injection of a fluid into an animal.

### BACKGROUND

In the keeping and rearing of animals, an injection often has to be administered to the animals. This can be carried out manually with a conventional syringe. In larger enterprises, for example in poultry farming, many animals are present, to which an injection is intended to be administered in as short a time as possible. There are therefore injection devices in the prior art, by means of which an injection to an animal can be carried out more rapidly.

For example, DE 10 2015 122 069 describes a device, in which a contact plate with an anatomically shaped contact surface is mounted displaceably on a basic body. The animal to which the injection is to be administered is pressed against the contact plate such that the latter is displaced in the direction of the basic body as far as an end position in which the injection is then administered. For this purpose, a syringe with a needle is moved through an opening in the contact surface and therefore pierced into the animal and the injection is then administered in said pierced state. Subsequently, the syringe with the needle is retracted again so that the needle no longer protrudes in front of the contact surface.

### SUMMARY

An improved device for the injection of a fluid into an animal is provided herein.

The device according certain embodiments for the needle-free injection of a fluid into an animal can comprise a contact device which comprises a shaped contact surface which is shaped in conformity with a body part of the animal to which the injection is to be administered. Furthermore, the device can comprise a first injector for the needle-free injection, which comprises a front dispensing end which, during the needle-free injection, extends through a first passage opening in the shaped contact surface and protrudes in relation to the shaped contact surface in a region adjacent to the first passage opening, a first measuring device which outputs at least one first measuring signal in order to detect contact of the animal with the front dispensing end of the first injector, and a control unit which activates (or triggers or actuates) a needle-free injection by means of the first injector on the basis of the at least one first measuring signal.

The desired needle-free injection for an animal can thus be carried out safely and reliably.

The control unit can decide in particular on the basis of the at least one first measuring signal whether there is contact of the animal with the front dispensing end of the first injector. If the control unit considers such contact to be given, it can then actuate the needle-free injection.

Contact between the animal and the front dispensing end of the first injector is understood as meaning in particular such contact which has to be present for a needle-free injection. This is preferably direct contact with the skin of the animal.

The measuring device can thus be designed in such a manner that it continuously outputs first measuring signals or that it outputs a first measuring signal when the contact between animal and front dispensing end of the first injector is achieved. In particular, the measuring device can carry out at least two different measurements and can transmit the first measuring signals generated in the process to the control unit.

The contact surface can be mounted displaceably between a main position and an injection position, wherein, in the main position of the contact surface, the front dispensing end of the first injector does not extend through the first passage opening in the shaped contact surface, and, by contrast, in the injection position of the contact surface, the front dispensing end of the first injector extends through the first passage opening in the shaped contact surface and protrudes in relation to the shaped contact surface in a region adjacent to the first passage opening.

The first measuring device can contactlessly detect the reaching of the triggering position.

The contact surface can be mounted in such a manner that the contact surface is in the main position without an animal pressed against it, and that the contact surface is movable into the injection position only upon a predetermined force being exceeded by the animal pressing against it.

In particular, the contact surface can be held in the main position under spring pretensioning.

The device for the needle-free injection of a fluid into an animal can comprise a first linear guide which carries the first injector, is mounted movably along a first displacement direction between a front end position and a triggering position and stops under spring pretensioning in the front end position, wherein the measuring device outputs a first measuring signal when the triggering position is reached by the first injector. The first linear guide is preferably designed in such a manner that the front dispensing end of the first injector, in the front end position, does not extend through the first passage opening in the shaped contact surface and does not protrude in relation to the shaped contact surface in a region adjacent to the first passage opening. Alternatively, it is however also possible for the front dispensing end of the first injector, in the front end position, to extend through the first passage opening in the shaped contact surface and to protrude in relation to the shaped contact surface in a region adjacent to the first passage opening.

The contact device can comprise in particular a contact plate which comprises the shaped contact surface.

The contact device can be positioned or mounted on a housing of the device. The contact device here can be designed and mounted on the housing in such a manner that the shaped contact surface is positionally fixed along the first displacement direction in the direction of the triggering position. In particular, the contact device can be arranged interchangeably or exchangeably.

The first measuring device can be designed in such a manner that it contactlessly detects the reaching of the triggering position.

Of course, it is also possible for the first measuring device to detect the reaching of the triggering position by means of detection of a contact.

The first linear guide can comprise a slide which is displaceable along the displacement direction and carries the first injector. The first measuring device can comprise a sensor for detecting the triggering position. In particular, the position of the slide can be measured for this purpose.

The sensor can be arranged at a position in the measuring arrangement that is fixed in position relative to the displacement movement of the slide. Alternatively, the sensor can be fastened to the slide itself and can therefore move together with the slide.

The sensor can be a proximity sensor or a distance sensor which carries out measurements by means of ultrasound, or magnetically, electrically, capacitively, inductively, mechanically and/or optically. In particular, the sensor can be a Hall sensor and therefore a sensor which uses a magnetic effect for the detection. Furthermore, for example, a potentiometer or a linear potentiometer can be used as the sensor, with which, for example, the slide (or an element connected in a fixed position to the slide) comes mechanically into contact when the triggering position is reached. The contact here may be fully present over a certain distance such that a precise measurement of the triggering position and/or a calibration for defining the triggering position is possible. Of course, it is also possible to design the sensor as a switch with which, for example, the slide (or an element connected in a fixed position to the slide) comes mechanically into contact when the triggering position is reached, the switch therefore supplying only a yes-no statement. The yes statement here designates that a contact is present, and thus the triggering position has been reached, whereas the no statement designates that no contact is present.

A proximity sensor is understood here as meaning in particular a sensor which outputs a switching signal after the first injector reaches the triggering position. A distance sensor is understood here as meaning in particular a sensor which outputs a signal which is proportional to the detected distance. It is therefore easily possible to correct and/or to individually adjust the position of the triggering position.

The first linear guide can comprise a first spring which holds the first injector in the front end position. The first spring can be configured in such a manner that a force of 5 to 10 Newtons has to be exerted on the front dispensing end in order to bring about a displacement of the injector counter to the spring force of the first spring.

The distance in the displacement direction between the front end position and the triggering position can be in the range of 5 to 10 mm. Therefore, by the animal pressing against the contact surface, a displacement of 5 to 10 mm has to be achieved. The needle-free injection is then carried out, should this be the case. It can therefore be ensured, for example, that the front dispensing end passes through the plumage into contact with the skin, e.g. in the region of the breast muscle, of the chicken.

The first linear guide can comprise a stop surface and a rod mounted on the slide, the rod being mounted displaceably parallel to the first displacement direction and being pretensioned toward the contact surface by means of a spring. A front end of the rod can lie against the stop surface when the triggering position is reached, and therefore the second spring acts upon the slide with a force toward the contact surface. This can be used, for example, in an advantageous way to provide an additional force (in addition to the force of the first spring) for the needle-free injection in order, for example, to compensate for a recoil occurring during the needle-free injection, and therefore the contact of the front dispensing end with the skin of the animal is securely maintained during the needle-free injection.

It is possible, for example, for a magnet (preferably a permanent magnet) to be arranged at the front end of the rod, the magnetic field of which magnet can be detected by means of the sensor in order to establish that the triggering position is reached.

In particular, the fluid can be present in liquid form or as a liquid. The fluid which is to be injected can be a medicament and/or a vaccine. In particular, the medicament and/or the vaccine can be administered as a liquid.

In the case of the device, the front dispensing end of the injector can furthermore extend through the first passage opening in the shaped contact surface and can protrude in relation to the shaped contact surface in a region adjacent to the first passage opening when the first injector is in the triggering position. It is therefore ensured that there is direct contact between the front dispensing end and that region of the animal into which the injection is intended to be performed.

The first injector can be embodied in particular in the form of a self-filling syringe. For this purpose, the first injector can comprise a supply port, to which a fluid reservoir can be connected, and therefore, after a needle-free injection, the first injector is filled again with fluid to be injected. The fluid reservoir can be, but does not have to be, part of the device for the needle-free injection.

The first measuring device can be designed in such a manner that it detects a force exerted on the first injector by the animal pressing against the contact surface, and thus against the front dispensing end of the first injector, and generates corresponding first measuring signals and transmits them to the control unit. An exceeding of a predetermined limit value can then be assessed by the control unit as contact between the animal and the front dispensing end of the first injector, and therefore a needle-free injection can be actuated.

Furthermore, the first measuring device can be designed in such a manner that it detects a force exerted on the contact surface by the animal pressing against the contact surface and generates corresponding first measuring signals and transmits them to the control unit which then triggers the needle-free injection for example when a predetermined limit value is exceeded.

Furthermore, the measuring device can comprise a proximity sensor (for example, a capacitive sensor) which detects the presence of the animal at the contact surface and outputs at least one first measuring signal. The proximity sensor can be positioned, for example, behind the contact surface, and therefore no direct contact between the animal and the sensor is possible, but the sensor can detect the presence of the animal at the contact surface.

The control unit is designed in particular in such a manner that it activates or triggers or actuates the needle-free injection by means of the first injector on the basis of a single first measuring signal or a plurality of first measuring signals (which are generated, for example, in the described manner by different measuring methods).

The first injector can comprise a piston-cylinder arrangement in which a spring-pretensioned piston rod is guided by its front end in a cylinder for receiving the fluid to be injected, the cylinder opening into a nozzle via a first nonreturn valve. Furthermore, a supply port can be provided which is connected to the cylinder via a second nonreturn valve. A fluid reservoir can be connected to the supply port.

The cylinder can be designed together with the first nonreturn valve and the nozzle (and preferably together with the second nonreturn valve and the supply port) as a releasable cylinder section in which a piston rod guide is arranged releasably at the end facing away from the nozzle. The piston rod guide can comprise a guide bushing, which is produced from plastic, for guiding the piston rod, and a metal fixing element connected to the guide bushing. In the installed state, the fixing element can be fixed (e.g. screwed) in the cylinder section, the fixing element having an engagement region for a tool, into which a tool can be inserted in order then to release (e.g. to unscrew) the fixing element from the cylinder section by means of the tool when the cylinder section is released from the rest of the injector.

Thus, on the one hand, good guidance of the piston rod by means of the guide bushing produced from plastic can advantageously be provided. On the other hand, the guide bushing can easily be removed and installed for maintenance purposes without being damaged since the engagement is undertaken for this purpose between the tool and the fixing element produced from metal.

The engagement region of the fixing element is preferably designed here in such a manner that the piston rod can extend through it without touching it.

The connection between the guide bushing and the fixing element is preferably separable without a tool.

For this purpose, for example, the connection between the guide bushing and the fixing element can be designed as a form-fitting connection.

The guide bushing can comprise a passage hole for guiding the piston rod. The passage hole can be hollow-cylindrical, for example. The cross section of the passage hole can be circular, for example.

The form-fitting connection between the guide bushing and the fixing element can be formed in particular in a direction parallel to the longitudinal direction of the passage hole. The form-fitting connection between the guide bushing and the fixing element can be formed, but does not have to be formed, transversely with respect to the longitudinal direction of the passage hole, wherein in this case there is preferably a lateral opening or recess in which there is no form-fitting connection, and therefore the guide bushing can be released or separated from the fixing element through said opening or recess.

The guide bushing and the fixing element are preferably connected releasably to each other. This is understood as meaning in particular that the connection of guide bushing and fixing element can be released without damaging guide bushing or fixing element.

The guide bushing can have a first annular groove on its outer side. The fixing element can have a laterally open collar with a lateral opening, with it being possible for the guide bushing to be pushed into the collar and out of the collar via the lateral opening in the collar. This is therefore a movement perpendicularly to the longitudinal extent of the guide bushing.

Therefore, the guide bushing can also be interchanged, if necessary.

The guide bushing can have a second annular groove on its outer side, in which a seal (e.g. a ring seal or an O-ring seal) sits.

Furthermore, the guide bushing can have an annular groove on its inner side, on which a second seal (e.g. a second ring seal or a second O-ring seal) sits.

The device can comprise a second injector and a second measuring device which are designed in the same way as the first injector and the first measuring device. The second injector can therefore comprise a front dispensing end which, during the needle-free injection, extends through a second passage opening in the shaped contact surface and protrudes in relation to the shaped contact surface in a region adjacent to the second passage opening. Furthermore, the second measuring device can output at least one second measuring signal in order to detect contact of the animal with the front dispensing end of the second injector, wherein the control unit activates a needle-free injection by means of the second injector on the basis of the at least one second measuring signal.

Furthermore, a second linear guide can be provided for the second injector, which is designed in the same way as the first linear guide for the first injector.

Said second injector can thus be mounted movably along a second displacement direction between a front end position and a triggering position and held under spring pretensioning in the front end position. The needle-free injection can then be performed in the same way as by means of the first injector if the movement of the second injector as far as the triggering position is detected by means of the second measuring device.

The first and second displacement directions can be parallel to each other or can enclose an angle with each other which is in the range between 0° and 45° and preferably smaller than 40°, 35°, 30° or 25° and preferably larger than 5°, 10°, 15° or 20°.

Therefore, two different fluids, e.g. medicaments and/or vaccines, can be simultaneously injected in the animal without a needle.

It goes without saying that the features mentioned above and the features yet to be explained below can be used not only in the specified combinations but also in other combinations or on their own without departing from the scope of the present invention.

The invention is explained in more detail below on the basis of exemplary embodiments with reference to the accompanying drawings, which also disclose features that are essential to the invention. These exemplary embodiments are provided for illustration only and should not be construed as limiting. For example, a description of an exemplary embodiment having a multiplicity of elements or components should not be construed as meaning that all of these elements or components are necessary for implementation. Rather, other exemplary embodiments may also contain alternative elements and components, fewer elements or components, or additional elements or components. Elements or components of different exemplary embodiments can be combined with one another, unless stated otherwise. Modifications and variations that are described for one of the exemplary embodiments can also be applicable to other exemplary embodiments. In order to avoid repetition, elements that are the same or correspond to one another in different figures are denoted by the same reference signs and are not explained repeatedly.

### BRIEF DESCRIPTION OF THE DRAWINGS

FIG. 1a shows a front view of an embodiment of the device for the needle-free injection of a fluid.

FIG. 1b shows a top view of the device from FIG. 1.

FIG. 1c shows a front view of the device from FIG. 1 with contact plate removed.

FIG. 2 shows an illustration of the rear side of the contact plate.

FIG. 3 shows an isometric view of the injector together with the linear guide.

FIG. 4 shows a sectional view of the cylinder-piston arrangement, in which the piston rod is in its front end position.

FIG. 5 shows a sectional view of the cylinder-piston arrangement, in which the piston rod is in its rear end position.

FIG. 6 shows an isometric exploded illustration of the injector together with the linear guide.

FIG. 7 shows a top view of the injector together with the linear guide.

FIG. 8 shows a sectional view of the injector with the linear guide along the intersecting line A-A in FIG. 7.

FIG. 9 shows an isometric exploded illustration of the linear guide.

FIG. 10 shows a top view of the linear guide.

FIG. 11 shows a sectional view of the linear guide along the intersecting line B-B from FIG. 10;

FIG. 12 shows a side view of the cylinder section;

FIG. 13 shows a sectional view of the cylinder section along the intersecting line C-C in FIG. 12;

FIG. 14 shows an isometric exploded illustration of the cylinder section.

FIG. 15 shows a side view of the piston rod guide.

FIG. 16 shows a sectional view of the piston rod guide along the intersecting line E-E in FIG. 15.

FIGS. 17 and 18 show isometric exploded illustrations of the piston rod guide.

FIG. 19 shows an isometric view of the injector together with the linear guide of a further embodiment of the device for the needle-free injection of a fluid.

FIG. 20 shows an isometric exploded illustration of the injector together with the linear guide from FIG. 19.

FIG. 21 shows a top view of the injector with the linear guide from FIG. 19.

FIG. 22 shows a sectional view of the injector with the linear guide along the intersecting line F-F in FIG. 21.

FIG. 23 shows an isometric exploded illustration of the linear guide from FIG. 19.

FIG. 24 shows a top view of the linear guide from FIG. 23.

FIG. 25 shows a sectional view of the linear guide along the sectional line G-G from FIG. 24.

FIG. 26 shows a sectional view of the linear guide along the sectional line H-H from FIG. 24.

FIG. 27 shows a front view of a further embodiment of the device for the needle-free injection of a fluid.

FIG. 28 shows a front view of the device from FIG. 26 with contact plate removed.

FIG. 29 shows a sectional view of the device along the intersecting line K-K from FIG. 27.

### DETAILED DESCRIPTION

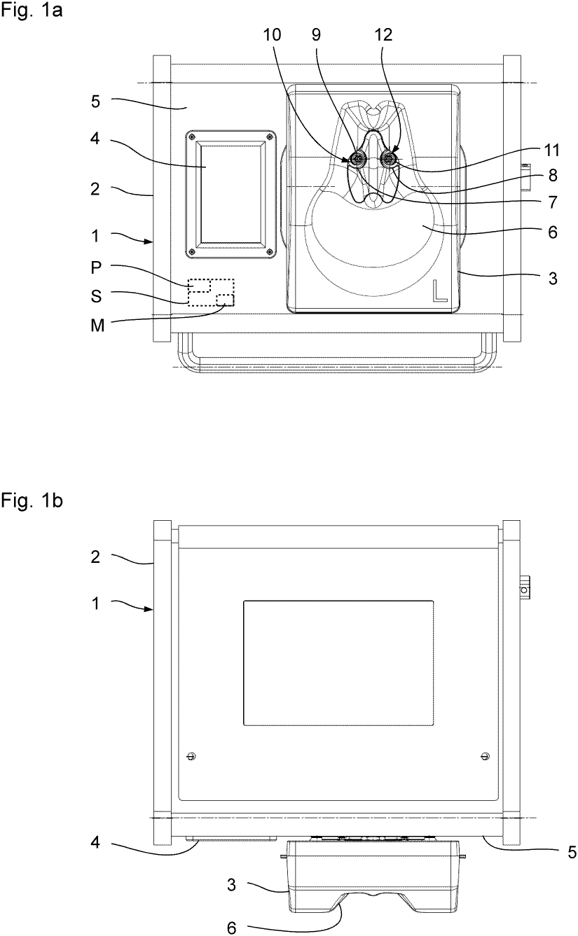

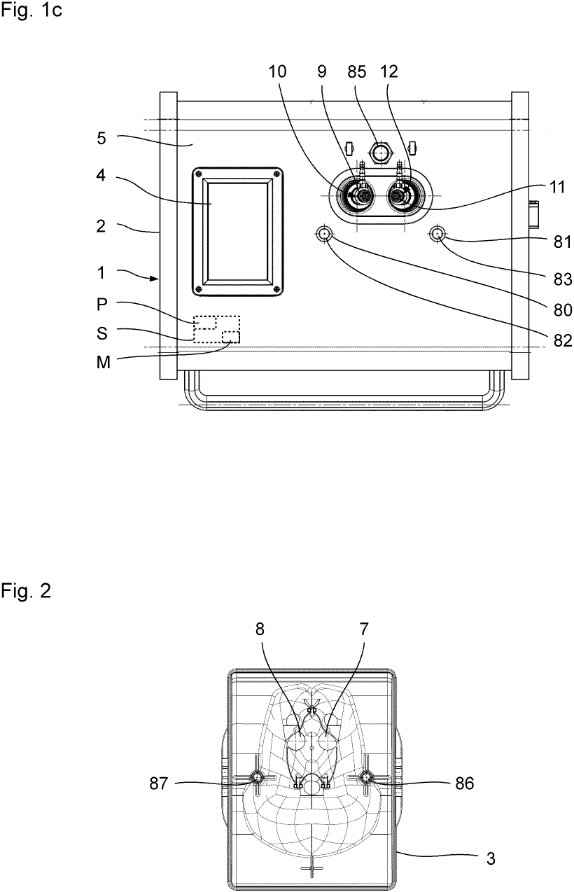

In the embodiment illustrated in FIGS. 1a - 1c and 2, the device 1 for the needle-free injection of a fluid comprises a housing 2 with a contact plate 3 and with a display 4 on a front side 5 of the housing 2. Furthermore, a control unit S arranged in the housing 2 with a processor P and a memory M for controlling the device 1 is provided, said control unit being illustrated merely schematically by dashed lines in FIGS. 1a and 1c.

The device 1 for the needle-free injection of a fluid serves for administering injections to an animal. With the embodiment illustrated, vaccines and/or medicaments can be administered intramuscularly, for example to a chicken.

For this purpose, the contact plate 3 has a shaped contact surface 6, the shape of which is selected in such a manner that it is anatomically matched to the animal to which the vaccine or the medicament is intended to be administered. In the case of the embodiment described here, the shaped contact surface 6 is shaped in conformity with the breast region of a chicken. In particular, the contact plate 3 can be provided removably on the front side 5. Successively different contact plates 3 can therefore be fitted which are matched, for example, to the type or breed of animal to be treated, to the age of the animals and/or to the size thereof.

As can readily be seen in particular in FIG. 1a, a first and a second passage opening 7, 8 are formed in the shaped contact surface 6. A front dispensing end 9 of a first needle-free injector 10 extends through the first passage opening 7 and a front dispensing end 11 of a second needle-free injector 12 extends through the second passage opening 8. The two dispensing ends 9, 11 therefore protrude in relation to those regions of the shaped contact surface 6 which directly surround the two passage openings 7, 8.

As can be gathered in particular from the illustration in FIG. 1c, a first and a second cylindrical support element 80, 81 are formed on the front side 5 of the housing 2 and extend substantially perpendicularly to the front side 5. The first cylindrical support element 80 comprises a first force sensor 82 and the second cylindrical support element 81 comprises a second force sensor 83. The force sensors can each be designed, for example, as a load cell known from the prior art.

Furthermore, a proximity sensor 85 is also arranged on the front side 5. The proximity sensor 85 can be embodied as a capacitive sensor 85, for example.

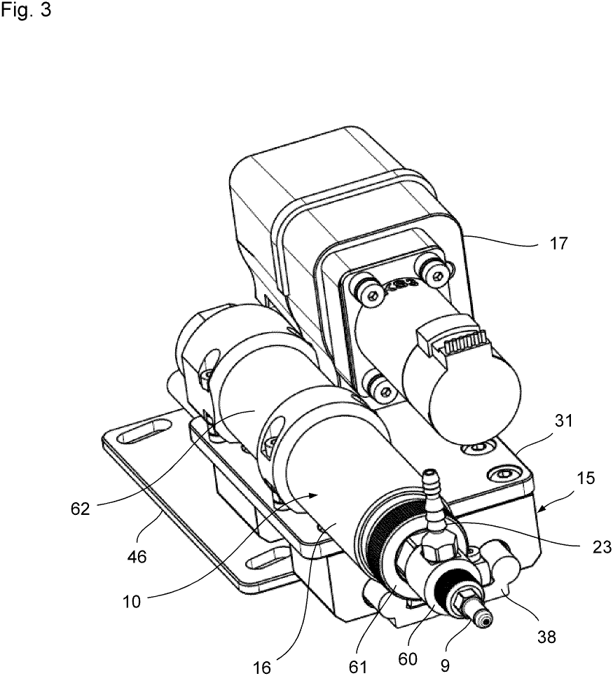

The two cylindrical support elements 80 and 81 serve for receiving and holding the contact plate 3 which, for this purpose, has two cylindrical cavities 86, 87 on its rear side, as shown in FIG. 2, such that the contact plate 3 can be pushed onto the two support elements 80, 81, which enter the cylindrical cavities 86, 87.

As can be gathered particularly readily from the illustration in FIG. 1c, the two cylindrical support elements 80, 81, and therefore the force sensors 82, 83 thereof, are arranged on the left and right of the two dispensing ends 9, 11 of the two injectors 10, 12. The proximity sensor 85 is arranged above the two dispensing ends 9, 11, and therefore the two force sensors 82 and 83 together with the proximity sensor 85 span a triangle which is an isosceles triangle here.

The arrangement of the two force sensors 82 and 83 makes it possible to measure the force with which the animal (here the chicken) is pressed against the contact plate 3. In particular, it is possible to detect whether the chicken is pressed obliquely or at a slant against it since, in this case, the two force sensors 82 and 83 display significantly different measuring values. The proximity sensor 85 also serves for measuring whether a chicken is lying sufficiently close to the contact plate 3. The force sensors 82 and 83 and the proximity sensor 85 can output measuring signals and transmit them to the control unit S. The control unit S can then evaluate these measuring signals and decide whether the chicken is lying correctly against the contact plate 3.

As will also be described in detail below, the two needle-free injectors 10, 12 are each mounted displaceably on a linear guide, with an injection being triggered only whenever the corresponding needle-free injector 10, 12 has been displaced by a predetermined distance. This displacement is achieved during correct use of the device 1 by the fact that the chicken is pressed with its breast region against the shaped contact surface 6 such that the front dispensing ends 9, 11 thereby come into contact through the plumage with the breast muscle (or the corresponding skin region) and are therefore pushed the predetermined distance rearward. It is therefore ensured that there is direct contact of the dispensing ends 9, 11 with the corresponding skin region at the breast muscle and then the needle-free injection can be triggered and performed successfully.

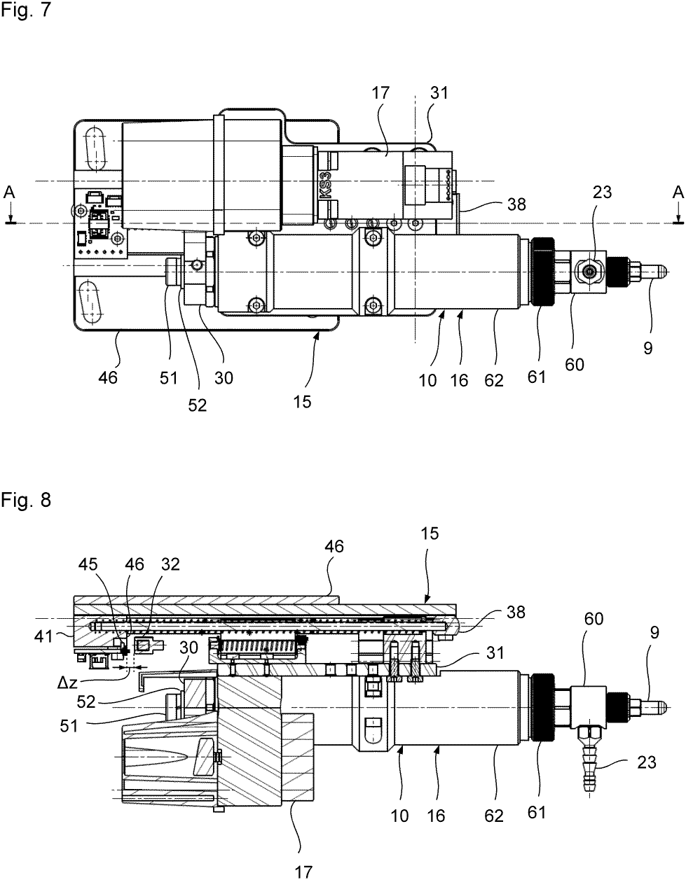

The first needle-free injector 10 together with the linear guide is described in more detail below with reference to FIGS. 3 to 11, the second needle-free injector 12 together with the linear guide being formed in the same manner. The first needle-free injector 10 is only called needle-free injector 10 below and comprises a piston-cylinder arrangement 16 and a tensioning device 17, which are fastened together on a linear guide 15 (FIG. 3).

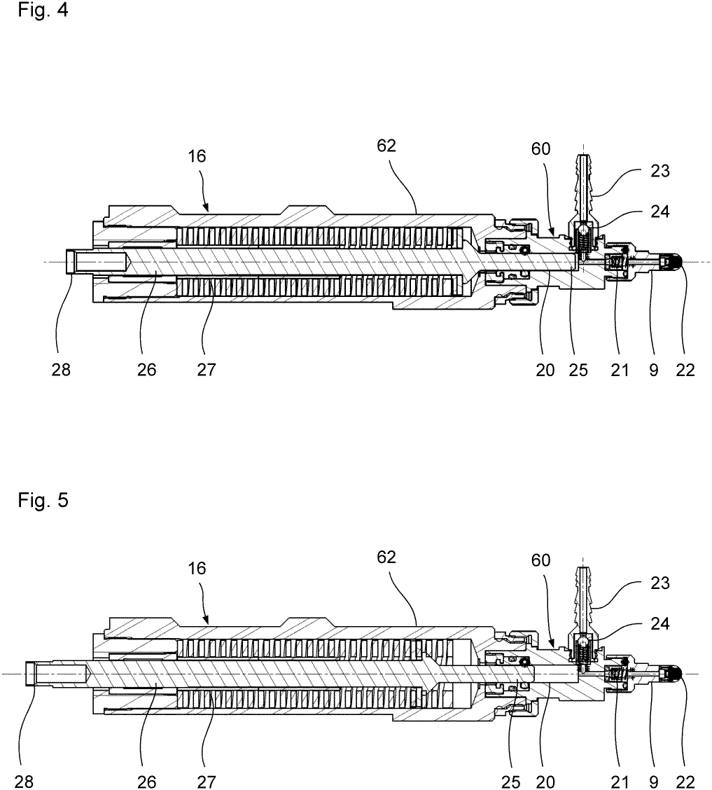

As can be gathered in particular from the sectional illustration in FIG. 4, the piston-cylinder arrangement 16 comprises a cylinder 20 which is fluidically connected to the front dispensing end 9, which comprises a nozzle 22 for dispensing the fluid, via a first nonreturn valve 21. The first nonreturn valve 21 is therefore designed in such a manner that fluid can be dispensed from the cylinder 20 via the first nonreturn valve 21 and the nozzle 22. It is not possible for air or liquid to be sucked up via the nozzle 22 and via the first nonreturn valve 21, since the first nonreturn valve 21 closes in this direction.

Furthermore, a fluid port 23 is provided which is fluidically connected to the cylinder 20 via a second nonreturn valve 24, and therefore fluid can pass into the cylinder 20 via the fluid port 23. The second nonreturn valve 24 provides a block in the opposite direction (i.e. from the cylinder 20 to the fluid port 23). The second nonreturn valve 24 can therefore be referred to as an inlet valve and the first nonreturn valve 21 can be referred to as an outlet valve.

A front end 25 of a piston rod 26 is guided in the cylinder 20, and therefore the front end 25 acts as a piston. The piston rod 26 is tensioned toward the front dispensing end 9 by a spiral spring 27 and has a rearwardly protruding end 28. The piston-cylinder arrangement 16 is therefore embodied as a self-filling syringe since a movement of the piston rod 26 from its front end position, shown in FIG. 4, counter to the spring force of the spiral spring 27, to the left in FIG. 4, leads to a negative pressure in the cylinder 20, as a result of which the second nonreturn valve 24 opens and fluid is sucked from a reservoir connected to the fluid port 23 into the cylinder 20. When the piston rod 26 has arrived in its rear end position (FIG. 5), the second nonreturn valve 24 closes and the cylinder 20 is filled with fluid. The piston-cylinder arrangement 16 is therefore in a pretensioned state. When the piston rod 26 is released, it accelerates toward the front dispensing end 9 because of the spiral spring 27, and therefore a positive pressure is built up in the cylinder 20, as a result of which the first nonreturn valve 21 opens and the fluid is dispensed via the nozzle 22 for the needle-free injection. This movement of the piston rod 26 ends when it reaches its front end position (FIG. 4). Renewed movement of the piston rod 26 to the rear end position counter to the spring force of the spiral spring 27 then leads again to filling of the cylinder 20 with fluid.

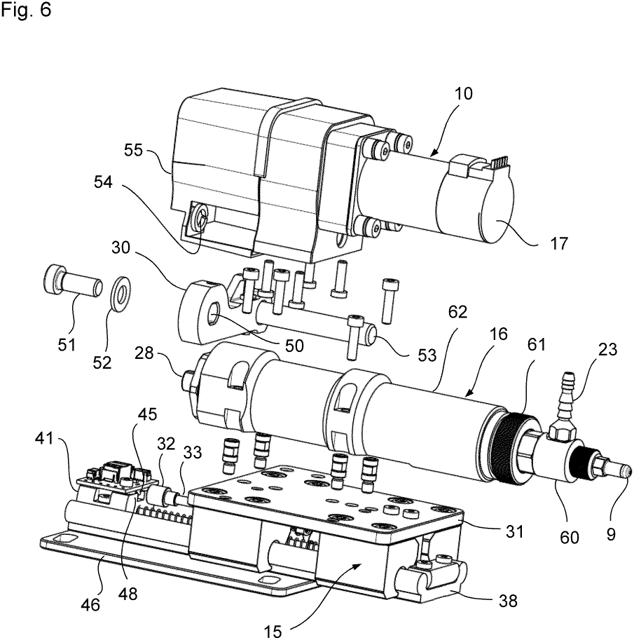

In order to carry out said described piston movement for filling purposes, a driver 30 (FIGS. 6-8) is fastened to the rear end 28 of the piston rod and can be moved by means of the tensioning device 17 in such a manner that the described tensioning of the piston rod 26, including the filling of the cylinder 20 with fluid, takes place, and the pretensioned state of the piston-cylinder arrangement 16 is maintained until the piston rod 26 is released for the needle-free injection. The driver 30 is fastened to the rear end 28 here through a through bore 50 in the driver 30 by means of a screw 51 and a plain washer 52.

To carry out such a tensioning movement, the tensioning device 17 can comprise, for example, a motor which sets a ramp track, which comprises a single winding, into rotation about an axis parallel to the longitudinal axis of the piston rod 26. A rotatable roller of the driver 30 is in contact with the ramp track, the driver 30 being mounted in the tensioning device 17 in such a manner that it is only movable parallel to the longitudinal axis of the piston rod 26 because of a guide rod 53, which is fastened to the driver 30 and is guided in a passage opening 54 on the motor housing 55. A rotational movement of the ramp track is therefore converted into a translational movement of the driver 30 parallel to the longitudinal axis of the piston rod 26. Such tensioning devices for devices for the needle-free administration are known. Reference is made here in particular to DE 10 2019 123 730 A1. Reference can be made here in particular to FIGS. 6 to 10 and the description in paragraphs 75 to 83.

By means of the rotation of the ramp track, which is caused by the motor, a movement of the piston rod 26 to the rear end position is therefore carried out. The ramp track has an upper plateau, which corresponds to the rear end position of the piston rod 26, and is held in said rotational position, as a result of which the pretensioned state of the piston-cylinder arrangement 16 according to FIG. 5 is present.

The upper plateau of the ramp track is connected to a lower plateau of the ramp track via a transition edge. If the ramp track is rotated further in order to carry out a needle-free injection, the roller runs over the transition edge, and therefore force is no longer present for tensioning the spring 27, and the piston rod 26 is thus abruptly accelerated toward the front dispensing end 9. Rotation of the ramp track as far as the upper plateau then leads to a next filling of the cylinder.

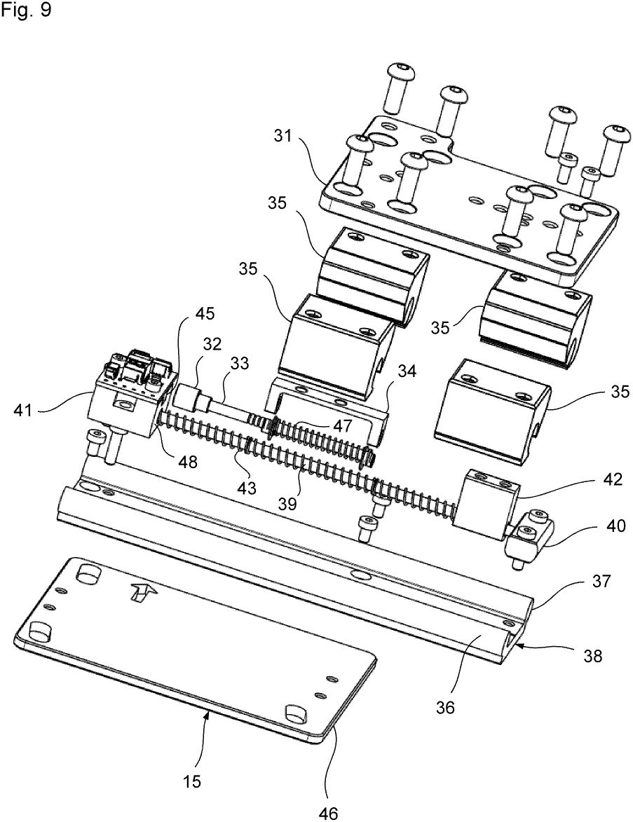

As can be gathered in particular from FIGS. 3 and 6 to 8, the piston-cylinder arrangement 16 is mounted together with the tensioning device 17 on a slide plate 31 of the linear guide 15. Furthermore, a magnet 32 is mounted on the slide plate 31 via a holding rod 33, and a holder 34 is mounted on the slide plate 31 (FIGS. 9-11). The slide plate 31 is guided via four guide blocks 35 on two guide rails 36, 37 of a guide base 38, and therefore the slide plate 31 can move only in the longitudinal direction (also called displacement direction below) of the guide rails 36, 37. In addition, the slide plate 31 is pretensioned in the direction of the contact plate 3 of the device 1 and is thereby in a front end position if an external force is not exerted on the front dispensing end 9 of the injector 10. For this purpose, a guide rod 39 is provided between two rod holders 40 and 41, with a spring stop 42 being guided on said guide rod and, for its part, being connected to the slide plate 31 and being pretensioned toward the contact plate 3 by means of a spring 43. The screws shown in the figures for connecting the parts will not be described in detail. Their functions are immediately apparent from the figures.

A sensor 45 (here a Hall sensor 45) is arranged on the second rod holder 41 and is used to detect the distance from the magnet 32.

The holding rod 33 is mounted displaceably in the displacement direction in the holder 34 and pretensioned toward the contact surface 6 parallel to the displacement direction by means of a second spring 47.

The guide base 38 is fastened on a base plate 46 which, for its part, is mounted in the housing 2.

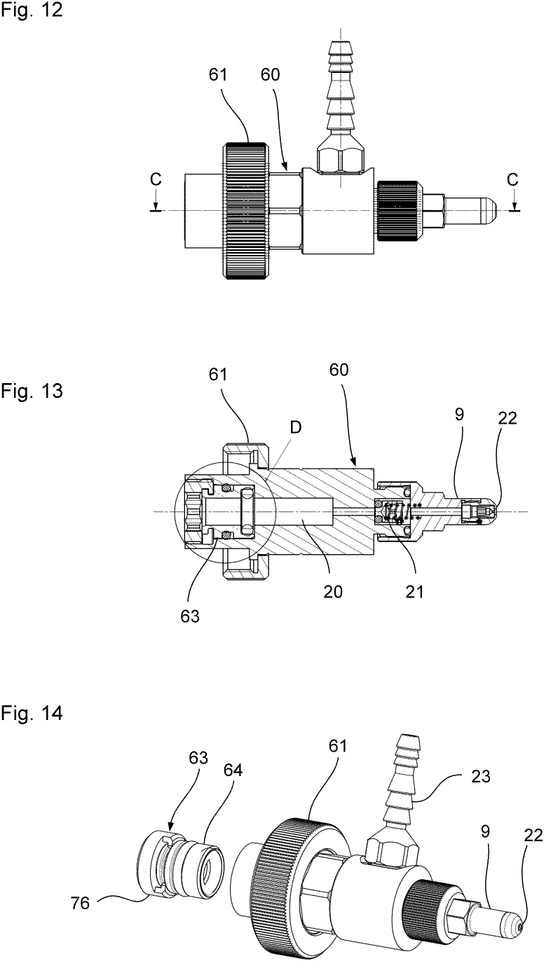

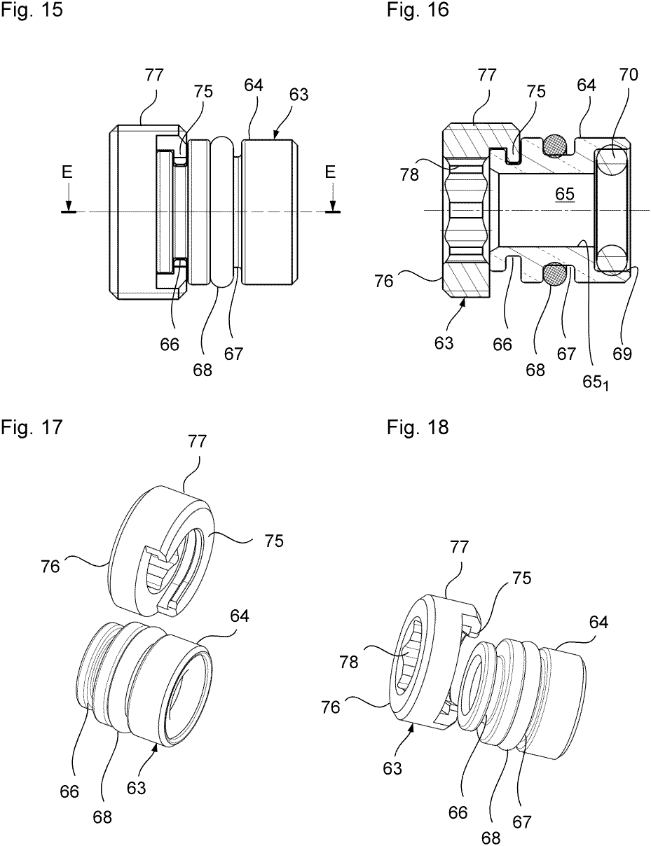

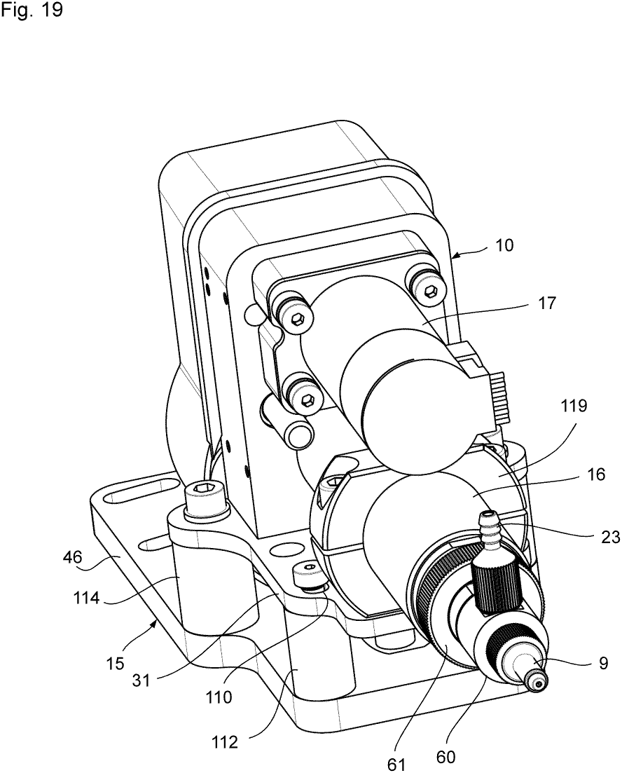

The cylinder 20 with the front dispensing end 9 and the fluid port 23 is designed as a releasable cylinder section 60 (FIGS. 12 and 13) which is fastened to a rear injector section 62 by a union nut 61 (FIGS. 3 to 8). To release the cylinder section 60, the contact plate 3 merely has to be removed, the union nut 61 released and then the cylinder section 60 can be pulled off from the rear injector section 62. As is apparent in particular in the sectional illustration in FIG. 13, the cylinder section 60 comprises a piston rod guide 63 (FIGS. 14 to 16) which, for its part, is fastened releasably in the cylinder section 60. The piston rod guide 63 comprises a guide bushing 64 which, in order to provide the necessary sliding properties for the piston rod 26, is formed from plastic and which has a passage hole 65, the inner wall 65₁ of which serves for guiding the front piston rod section 25. The guide bushing 64 has a first annular groove 66 and a second annular groove 67 on its outer side. A first O-ring seal 68 sits in the second annular groove 67. Furthermore, the guide bushing 64 comprises a third annular groove 69 on the inner side. A second O-ring seal 70 sits in the third annular groove 69.

Since the O-ring seals 68 and 70 wear, they have to be frequently exchanged. For this purpose, the piston rod guide 63 has to be able to be removed from the cylinder section 60. Since the guide bushing 64 is fixed relatively strongly in the cylinder section 66 in order to ensure permanent operation, direct action on the guide bushing 64 with a tool for removal of the guide bushing 64 would lead to the latter being damaged.

The guide bushing 64 therefore fits with its first annular groove 66 in a laterally open collar 75 of a fixing element 76 with an external thread 77 and a hexagon socket 78. An internal thread matching the external thread 77 is provided in the corresponding receptacle of the cylinder section 60, and therefore the fixing element 76 can be screwed into the cylinder section 60 and also can be unscrewed again with a corresponding tool (e.g. hexagon key) which engages in the hexagon socket 78. Both the fixing element 76 and the cylinder section 60 are preferably formed from metal. As soon as the fixing element 76 is unscrewed, the entire piston rod guide 63 can therefore be removed so that the O-ring seals 68 and 70 can be changed.

Since the guide bushing 64 sits in the laterally open collar 75, there is a form-fitting connection in the longitudinal direction of the passage hole 65. In a direction transversely with respect to the longitudinal direction of the passage hole 65, the form-fitting connection is undone laterally only through the open region of the collar 75, and therefore the guide bushing can be pushed out laterally through the open region of the collar 75 and therefore transversely with respect to the longitudinal direction of the passage hole 65. It is therefore also possible to release the guide bushing 65 in the described manner from the fixing element 76 and then to exchange the O-ring seals 68 and 70. The guide bushing 64 with the exchanged O-ring seals 68 and 70 can then be pushed again laterally into the collar 75, and the entire piston rod guide 63 can then in turn be screwed into the cylinder section 60 in the described manner.

The wearing parts (here the O-ring seals 68, 70) can therefore be easily exchanged without the guide bushing 64 being damaged in the process.

Furthermore, it is possible to exchange the guide bushing 64 itself should this be necessary. The exchanged guide bushing 64 can then be fixed in the cylinder section 60 by means of the fixing element 76 without being damaged.

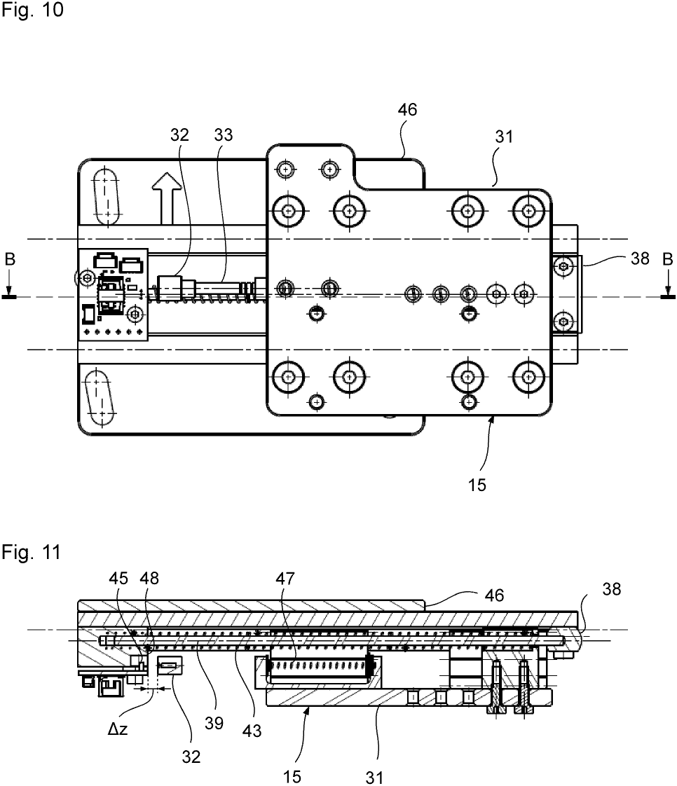

When the device 1 for the needle-free injection of a fluid is used correctly, the needle-free injector 10 is spring pretensioned on the basis of the linear guide 15 (because of the first spring 43) in its front end position in which the front dispensing end 9 extends through the first passage opening 7. If the animal (here a chicken with its breast region) is now pressed against the shaped contact surface 6, the front dispensing end 9 thereby comes into contact through the plumage with the breast muscle (or the corresponding skin region). Owing to the linear guide 15, a pressure on the front dispensing end 9 of the needle-free injector 10 leads to a movement of the needle-free injector 10 with the slide plate 31 in the direction of the second rod holder 41 counter to the force of the first spring 43. If the magnet 32 comes into contact with the second rod holder 41 (or a stop surface 48 of the second rod holder 41), the needle-free injector 10 is in its triggering position. The distance Δz covered in the process between the magnet 32 and the stop surface 48 of the second rod holder 41 (FIGS. 8 and 11) can be in the range from 5 - 10 mm. Furthermore, by means of this linear movement into the triggering position, the distance from the sensor 45 has been reduced such that the latter outputs a triggering signal or a corresponding measuring signal, which indicates that the triggering position is reached, to the control unit S. The control unit S then actuates the tensioning device 17 to release the pretensioned piston rod 26 so that the desired needle-free injection is performed. Since the sensor 45 is set back somewhat from the stop surface 48, there is no direct contact between the sensor 45 and the magnet 32 even in the triggering position.

The control unit S can actuate the needle-free injection on the basis only of the measuring signal of the sensor 45. However, it is advantageous if, for this purpose, the control unit S additionally evaluates the measuring signals of the two force sensors 82 and 83 and of the proximity sensor 85. It can readily be detected by the two force sensors 82 and 83 that the chicken is pressed in the conceived way against the contact surface 6. Should the chicken be positioned obliquely against the contact surface 6, the values of the two force sensors 82 and 83 would differ by a predetermined value which would indicate that a needle-free injection cannot be ensured. Furthermore, the measuring signal of the proximity sensor 85 can also be taken into consideration so that there is a greater degree of certainty that the chicken is lying in the predetermined manner against the contact surface 6 and is pressed against the latter.

In the case of the needle-free injection by means of the needle-free injector 10, the design causes a certain recoil in the needle-free injector 10, which could lead to the desired direct contact between the front dispensing end 9 and the breast muscle (or the corresponding skin region) being released, which may lead to a poorer needle-free injection. Therefore, the second spring 47 is arranged on the holding rod 33 in the holder 34, said spring, because of the contact between the magnet 32 and the stop surface 48, exerting an additional counterforce on the slide plate 31 and pressing the latter toward the front dispensing end 9 (and therefore toward the contact surface 6). The force of the second spring 47 is therefore connected in the triggering position to the force of the first spring 43, and therefore the recoil of the needle-free injector 10 during the injection is compensated for and thus the desired direct contact between the front dispensing end 9 and the breast muscle (or the corresponding skin region) is maintained.

The needle-free injection is followed in turn by the pretensioning of the piston rod 26 and maintaining of the pretensioned state. However, a renewed needle-free injection can be performed again only if the distance between the magnet 32 and the sensor 45 corresponds again to the value when no pressure is exerted on the front dispensing end 9 of the needle-free injector 10 (this corresponds to the removal of the animal from the contact plate 3). It can thereby be ensured that the particular animal is injected without a needle with the vaccine or the medicament just once.

The two needle-free injectors 10, 12 are provided in the described embodiment, and therefore two vaccines and/or medicaments can be injected simultaneously without a needle.

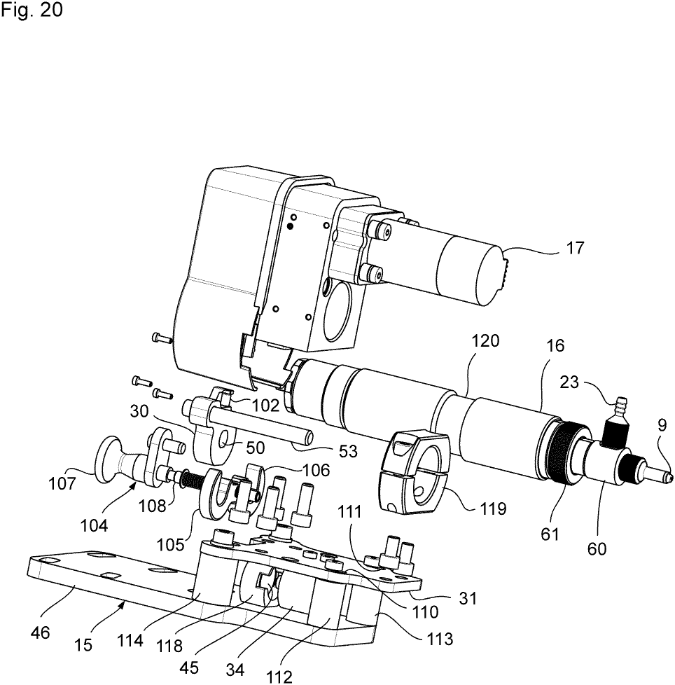

A further embodiment of the device 1 for the needle-free injection of a fluid is shown in FIGS. 19 - 23, with essentially the design of the linear guide 15 and the arrangement of the piston-cylinder arrangement 16 with respect to the tensioning device 17 differing in comparison to the previously described embodiment. The details will be described below.

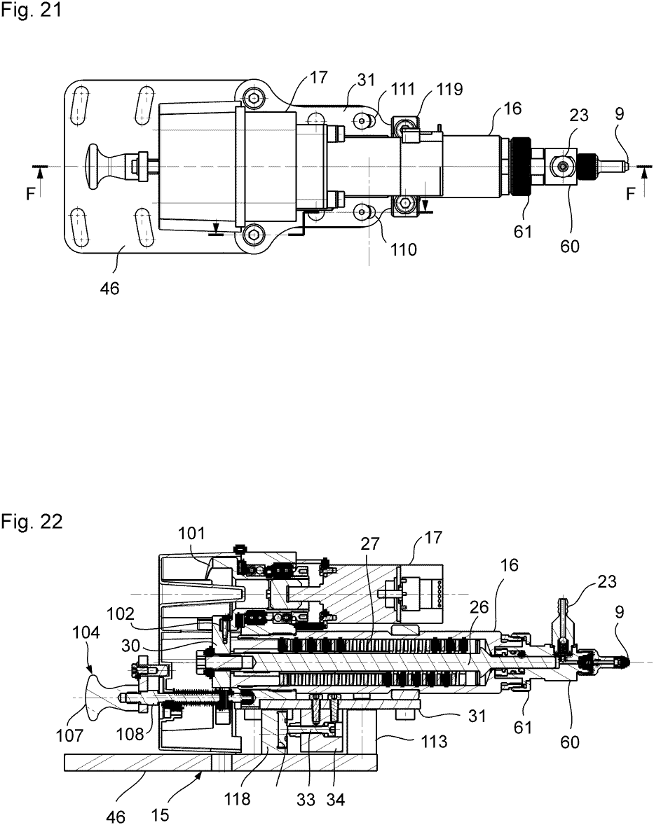

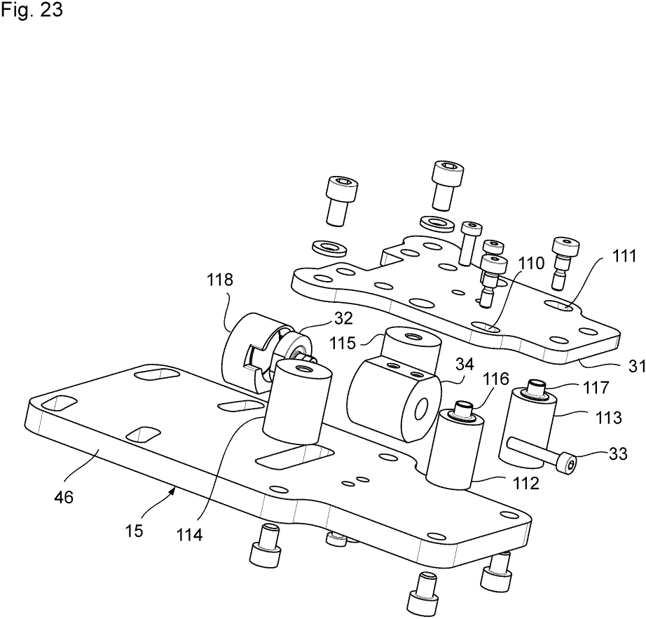

As can be gathered in particular from FIGS. 19 - 21, the tensioning device 17 is arranged above the piston-cylinder arrangement 16 (with respect to the slide plate 31). This leads to a somewhat modified design of the driver 30, its roller 102 being visible in FIGS. 20 and 21 and the ramp track 101 of the tensioning device 17 being visible in FIG. 22.

In addition, a dose setting unit 104 with a first and a second spacer 105, 106 which can be pivoted in and are each U-shaped is provided (FIG. 20). The dose setting unit 104 comprises a rotary button 107 which is connected to one end of an axis of rotation 108, at the other end of which the two spacers 105, 106 are connected for rotation therewith. Rotation of the rotary button 107 therefore leads to pivoting of the two spacers 105, 106. In the neutral rotational position of the rotary button 107, which is shown in FIGS. 20 and 21, and therefore the rotational position of the two spacers 105, 106, neither of the two spacers 105, 106 is between the driver 30 and the stop of the driver 30 at the rear end of the piston-cylinder arrangement 16. The maximum stroke of the piston 25 is therefore present (the piston rod is in its rear end position), said stroke being determined by the upper plateau of the ramp track 101.

If the ramp track 101 is rotated in order to carry out a needle-free injection, the roller 102 runs over the transition edge, and therefore force is no longer present for tensioning the spring 27, and the piston rod 26 is thus abruptly accelerated toward the front dispensing end 9 until the driver 30 lies against the rear end of the piston-cylinder arrangement 16. Rotation of the ramp track as far as the upper plateau then leads to a next filling of the cylinder.

In this state, by rotation of the rotary knob 107, the first or second spacer 105, 106 can then be positioned between the driver 30 and the rear end of the piston-cylinder arrangement 16. The extent of the first or second spacer 105, 106 in the longitudinal direction of the piston rod 26 then corresponds to the reduction of the stroke during the needle-free injection since, during the needle-free injection, the movement of the piston rod 26 is stopped upon contact of the driver 30 with the first or second spacer 105, 106. This leads to a lower amount of fluid (or lower volume of fluid) which is dispensed during the needle-free application. Since the first spacer 105 has a different extent here in the longitudinal direction of the piston rod 26 than the second spacer 106, two different amounts of fluid can be set by means of the spacers 105, 106, both amounts of fluid being smaller than the amount of fluid at the maximum stroke. Three different amounts of fluid can therefore be dispensed with the described device 1 for the needle-free injection of a fluid.

The first spacer 105 can comprise two, three or more sections (not shown) in the circumferential direction which each have a different extent in the longitudinal direction of the piston rod 26. The same applies to the second spacer 106. More than three different amounts of fluid can therefore be set. It is also possible for only the first or second spacer to be provided.

The dose setting unit 104 can be designed in such a manner that it can be operated manually, as is the case in the described embodiment. The user merely has to rotate the rotary button 107. Of course, the dose setting unit 104 can also be designed in such a manner that the rotation of the axis of rotation is motor-driven.

The linear guide 15 comprises the slide plate 15 and the base plate 46, the slide plate 15 having two elongated holes 110, 111 which permit the movement of the slide plate 15 in the longitudinal direction or in the displacement direction. For this purpose, the slide plate 15 is connected to the base plate 46 via two rigid cylinders 112, 113 and two elastic cylinders 114, 115.

The rigid cylinders 112, 113, which here are metal cylinders, each have a cylindrical guide pin 116, 117 which is placed into the corresponding elongated hole 110, 111 and the outside diameter of which is selected in such a manner that a relative movement between guide pin 116, 117 and elongated hole 110, 111 in the displacement direction is possible.

The elastic cylinders 114, 115 are of such elasticity that an elastic deformation is possible under customary forces against the front end 9 when an animal is pressed against the contact surface 6 of the contact plate 3, and therefore the slide plate 31 moves relative to the base plate 46 in the displacement direction because of being guided by the elongated holes 110, 111.

To detect this displacement of the slide plate 31, the holder 34 with the holding rod 33 and the magnet 32 is mounted on the bottom side of the slide plate 31. A sensor holder 118, in which the sensor 45 (here, e.g., the Hall sensor 45) is arranged, is mounted on the base plate 46. The cylinders 112-115 are screwed to the slide plate 31 and the base plate 46 by the screws in the figures (e.g. FIG. 23). The fastening of the holder 34 to the slide plate 31 and of the sensor holder 118 to the base plate 46 is also undertaken by screws. These screws are illustrated in FIGS. 19 - 23, but are not provided with reference signs. Of course, any other type of fastening or connection is also possible. The piston-cylinder arrangement 16 is fastened to the slide plate 31, for example, by means of a sleeve 119 (e.g. a clamping sleeve) which is screwed to the slide plate 31 and is in engagement with an outer groove 120 on the piston-cylinder arrangement 16.

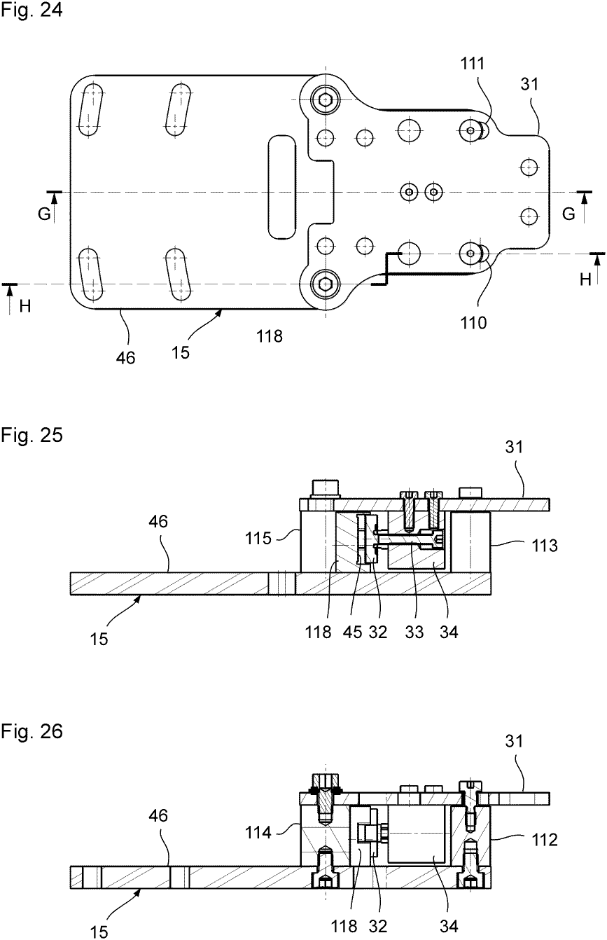

When the device 1 for the needle-free injection of a fluid is used correctly, the needle-free injector 10 is spring-pretensioned on the basis of the linear guide 15 (because of the elastic cylinders 114, 115) in its front end position (e.g. shown in FIG. 24) in which the front dispensing end 9 extends through the first passage opening 7. If the animal (here a chicken with its breast region) is now pressed against the shaped contact surface 6, the front dispensing end 9 thereby comes into contact through the plumage with the breast muscle (or the corresponding skin region). Owing to the linear guide 15, a pressure on the front dispensing end 9 of the needle-free injector 10 leads to a movement of the needle-free injector 10 with the slide plate 31 in the direction of the sensor holder 118 counter to the spring force of the two elastic cylinders 114, 115. A displacement of the slide plate 31 therefore leads to a movement of the magnet 32 in the displacement direction, as a result of which the distance between magnet 32 and sensor 45 becomes smaller. If the rear stop of the slide plate 31 is reached in the displacement direction (defined by the extent of the elongated hole 110, 111 in the displacement direction), there is a distance between magnet 32 and sensor 45, which leads to a sensor signal that is interpreted as the animal lying against it. This position of the slide plate 31 can also be referred to as the triggering position. The needle-free injection can then be performed in the described manner.

The triggering position has to be the rear stop of the slide plate 31. A position in front of the rear stop can also be determined as the triggering position. It should merely be ensured that there is the desired contact between the skin of the animal (here breast muscle of the chicken) and the front dispensing end 9 in order to be able to perform the desired needle-free injection.

Instead of the described measurement by means of the Hall sensor 45, it is also possible for a force sensor to be provided which then measures the force by which the corresponding end 32 of the holding rod 33 is pressed against the force sensor 45. In this case, the end 32 does not have to have a magnet.

Of course, in this embodiment too, the measuring signals of the two force sensors 82, 83 and the proximity sensor 85 can also be evaluated in the described way in order to perform the needle-free injection.

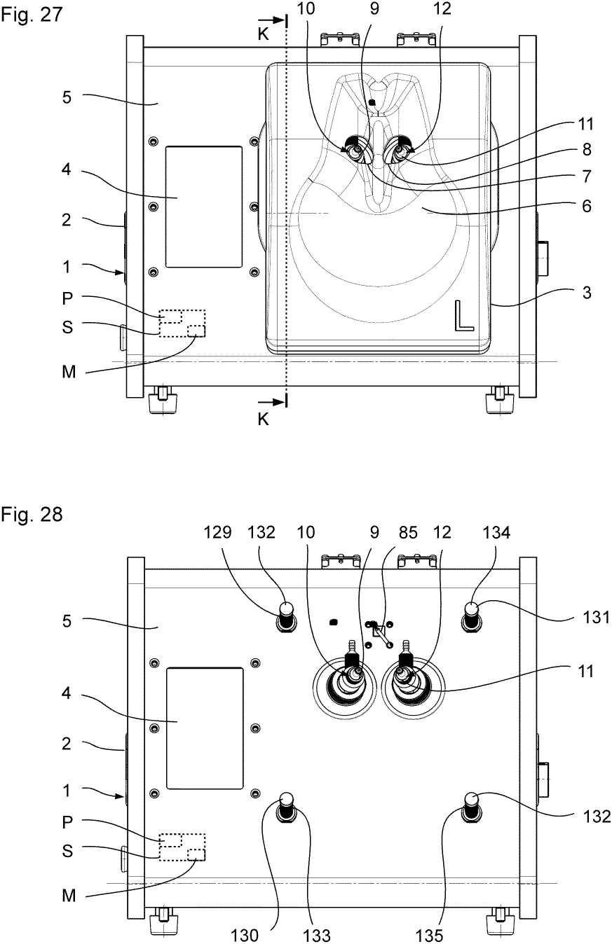

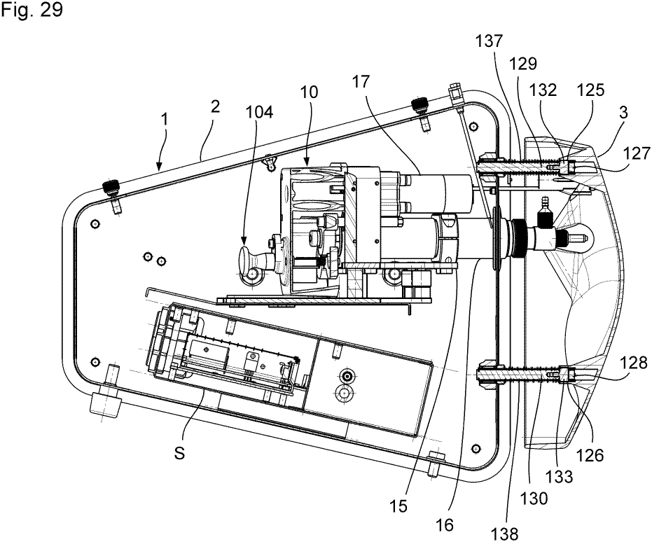

In the case of the previously described embodiments, the contact plate 3 is mounted on the housing 2 in such a manner that it cannot be displaced in the displacement direction. However, it is possible to mount the contact plate 3 on the housing 2 so as to be displaceable in the displacement direction, as is shown, for example, in FIGS. 27 - 29.

On its side facing the housing 2, the contact plate 3 comprises four hollow-cylindrical receptacles (two receptacles 125, 126 are visible in the sectional view of FIG. 29), in each of which a magnet 127, 128 sits. From the front side 5 of the housing 2, four guide rods 129, 130, 131, 132 protrude which are inserted into the four hollow-cylindrical receptacles 125, 126. Magnets 133, 134, 135, 136 are arranged at the free ends of the guide rods 129, 130, 131, 132 facing the contact plate 3, and therefore the contact plate 3 which is pushed onto the guide rods 129, 130, 131, 132 is magnetically fixed.

The four guide rods 129, 130, 131, 132 are each mounted displaceably with respect to the front side 5 of the housing 2 in such a manner that they can be pushed into the housing 2. However, the four guide rods 129, 130, 131, 132 are each pretensioned by a spring 137, 138 in the direction of the contact plate 3 in such a manner that the contact plate 3, if an animal is not pressed against the contact plate 3, is in its basic position which is shown in FIG. 29. In this basic position, the front ends 9, 11 of the two needle-free injectors 10, 12 do not extend through the two passage openings 7, 8 in the shaped contact surface 6 and therefore do not protrude in relation to the shaped contact surface 6 in a region adjacent to the two passage openings 7, 8.

If an animal is now pressed against the contact plate 3, the contact plate 3 is thereby moved in the direction of the front side 5 (the guide rods 129, 130, 131, 132 enter the housing 2) until the contact plate 3 lies against the front side 5. In this injection position of the contact plate 3, the front ends 9, 11 of the two needle-free injectors 10, 12 protrude in relation to the shaped contact surface 6 such that there is also already contact between the breast muscle and the front dispensing ends 9, 11, which contact has moved the needle-free injectors as far as the triggering position because of the described linear guide 15. The needle-free injection is thus performed, with it being ensured that the chicken is positioned correctly.

The described contact plate 3 is preferably designed in such a manner that all of the parts of the contact plate 3 move in the same way if an animal is pressed against it. In particular, the contact plate 3 can be formed as a single part.

The contact plate 3 may also be formed, for example, in two parts, with an inner part which forms at least part of the shaped contact surface 6 being displaceable in the displacement direction in relation to an outer part of the contact plate 3. This can be designed in particular as is described in DE 10 2015 122 069 A1. Reference is made in particular to FIGS. 1 - 5 and the description in paragraphs 81 - 93 of DE 10 2015 122 069 A1.

What is claimed is:

1. A device for the needle-free injection of a fluid into an animal, comprising:
a contact device, comprising a shaped contact surface that is shaped in conformity with a body part of the animal to which the injection is to be administered;
a first injector for the needle-free injection, comprising a front dispensing end which, during the needle-free injection, extends through a first passage opening in the shaped contact surface and protrudes in relation to the shaped contact surface in a region adjacent to the first passage opening;
a first measuring device that outputs at least one first measuring signal in order to detect contact of the animal with the front dispensing end of the first injector; and
a control unit that activates a needle-free injection via the first injector on the basis of the at least one first measuring signal.

2. The device of claim 1, further comprising a first linear guide that carries the first injector, first linear guide mounted movably along a first displacement direction between a front end position and a triggering position and stops under spring pretensioning in the front end position, wherein the first measuring device outputs a first measuring signal when the triggering position is reached by the first injector.

3. The device of claim 2, wherein the first measuring device contactlessly detects the reaching of the triggering position.

4. The device of claim 2, wherein the contact surface is mounted displaceably between a main position and an injection position, wherein, in the main position of the contact surface, the front dispensing end of the first injector does not extend through the first passage opening in the shaped contact surface, and, in the injection position of the contact surface, the front dispensing end of the first injector extends through the first passage opening in the shaped contact surface and protrudes in relation to the shaped contact surface in a region adjacent to the first passage opening.

5. The device of claim 4, wherein the contact surface is mounted in such a manner that the contact surface is in the main position without an animal pressed against it, and that the contact surface is movable into the injection position only upon a predetermined force being exceeded by the animal pressing against it.

6. The device of claim 4, wherein the contact surface is held in the main position under spring pretensioning.

7. The device of claim 1, wherein the contact surface is mounted displaceably between a main position and an injection position, wherein, in the main position of the contact surface, the front dispensing end of the first injector does not extend through the first passage opening in the shaped contact surface, and, in the injection position of the contact surface, the front dispensing end of the first injector extends through the first passage opening in the shaped contact surface and protrudes in relation to the shaped contact surface in a region adjacent to the first passage opening.

8. The device of claim 7, wherein the contact surface is mounted in such a manner that the contact surface is in the main position without an animal pressed against it, and that the contact surface is movable into the injection position only upon a predetermined force being exceeded by the animal pressing against it.

9. The device of claim 7, wherein the contact surface is held in the main position under spring pretensioning.

10. The device of claim 1, wherein the fluid volume dispensed during the needle-free injection is adjustable.

11. The device of claim 1, wherein the first measuring device detects a force exerted on the first injector by the animal pressing against the contact surface, and thus against the front dispensing end of the first injector, and outputs first measuring signals to the control unit which activates the needle-free injection when a predetermined limit value is exceeded.

12. The device of claim 1, wherein the first measuring device detects a force exerted on the contact surface by the animal pressing against the contact surface and outputs first measuring signals to the control unit which triggers the needle-free injection when a predetermined limit value is exceeded.

13. The device of claim 1, wherein the first measuring device comprises a proximity sensor which detects the presence of the animal at the contact surface and outputs a first measuring signal to the control unit which triggers the needle-free injection on the basis of the first measuring signal of the proximity sensor.

14. The device of claim 1, wherein the first injector is a self-filling syringe.

15. The device of claim 1, further comprising:
a second injector for the needle-free injection, which comprises a front dispensing end which, during the needle-free injection, extends through a second passage opening in the shaped contact surface and protrudes in relation to the shaped contact surface in a region adjacent to the second passage opening, and
a second measuring device which outputs at least one second measuring signal in order to detect contact of the animal with the front dispensing end of the second injector, wherein the control unit activates a needle-free injection via the second injector on the basis of the at least one second measuring signal.
